(19) United States
(12) Patent Application Publication
(10) Pub. No.: US 2024/0182926 A1
(43) Pub. Date: Jun. 6, 2024

(54) Nanofiber- and Nanowhisker-Based Transfection Platforms for Bulk Electroporation

(71) Applicants: Ohio State Innovation Foundation, Columbus, OH (US); Nanofiber Solutions, LLC, Hilliard, OH (US)

(72) Inventors: Daniel Gallego-Perez, Columbus, OH (US); Natalia Higuita-Castro, Columbus, OH (US); Jed Johnson, Hilliard, OH (US); Devleena Das, Columbus, OH (US); Silvia M. Duarte-Sanmiguel, Columbus, OH (US)

(21) Appl. No.: 18/549,702

(22) PCT Filed: Apr. 13, 2022
(86) PCT No.: PCT/US2022/024632

§ 371 Date: Sep. 8, 2023

Related U.S. Application Data
(60) Provisional application No. 63/177,613, filed on Apr. 21, 2021.

(51) Int. Cl. C12N 15/87; B82Y 5/00; B82Y 30/00; C12N 13/00; D01F 4/00; D01F 6/62

(52) U.S. Cl. CPC C12N 15/87; C12N 13/00; D01F 4/00; D01F 6/625; B82Y 5/00; B82Y 30/00; D10B 2211/06; D10B 2211/20; D10B 2401/12; D10B 2401/16

(57) ABSTRACT

Described herein are methods of using electrospun core-shell fibers for bulk electroporation. The disclosed electrospun core-shell fibers include (i) a central core that is electrically conductive having an exterior surface, wherein the core comprises a first polymer and an electroconductive material; (ii) a shell adjacent to the exterior surface of the core, the shell comprising a second polymer; and (iii) one or more bioactive agents in the shell. In one aspect, the fibers are electrospun fibers.

### CROSS-REFERENCE TO RELATED APPLICATIONS

This application claims benefit of U.S. Provisional Application No. 63/177,613, filed Apr. 21, 2021, which is hereby incorporated herein by reference in its entirety.

### BACKGROUND

Conventional electroporation methods often result in low cell viability due to heat generation (especially with primary cells). These methods can also allow for non-specific transport of molecules into/out of cells, and result in a high number of vector integrations, which can lead to mutagenesis because insertions are essentially random. Furthermore, electroporation tends to be much less effective for DNA insertion (when compared to RNA insertion), because the material must cross two phospholipid bilayer membranes (the cell membrane and the nuclear membrane). Some commercial flow electroporation systems offer higher cellular viability rates and greater efficiency than conventional electroporation systems while maintaining throughput, but still perform poorly for electroporation of primary cells and DNA insertion.

### SUMMARY

Described herein are methods of using electrospun core-shell fibers for ex vivo delivery of cargo (e.g., nucleic acids, proteins) to cells. The disclosed electrospun core-shell fibers include (i) a central core that is electrically conductive having an exterior surface, wherein the core comprises a first polymer and an electroconductive material; (ii) a shell adjacent to the exterior surface of the core, the shell comprising a second polymer; and (iii) one or more bioactive agents in the shell. In one aspect, the fibers are electrospun fibers.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 demonstrates the existence of nanochannels on the fiber surface.

FIG. 2 demonstrates that nanofibers can also be micronized into a nanowhisker format.

FIGS. 3A-3C depict schematics of exemplary core-shell fibers according to various aspects of the disclosure including either a solid biodegradable shell (FIG. 3B and left figure in FIG. 3A) or a nanostructured or nanoporous shell (FIG. 3C and right figure in FIG. 3A).

FIG. 4 shows cells incubated with GFP plasmid.

FIG. 5 shows cells incubated with fibers with no GFP plasmid coating/shell.

FIG. 6 shows cells incubated with GFP plasmid coating/shell.

FIG. 7 shows cells incubated with GFP plasmid.

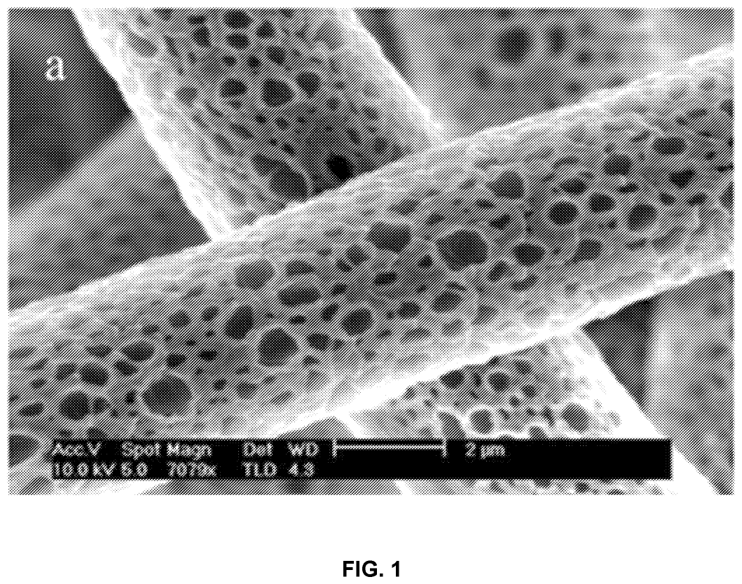

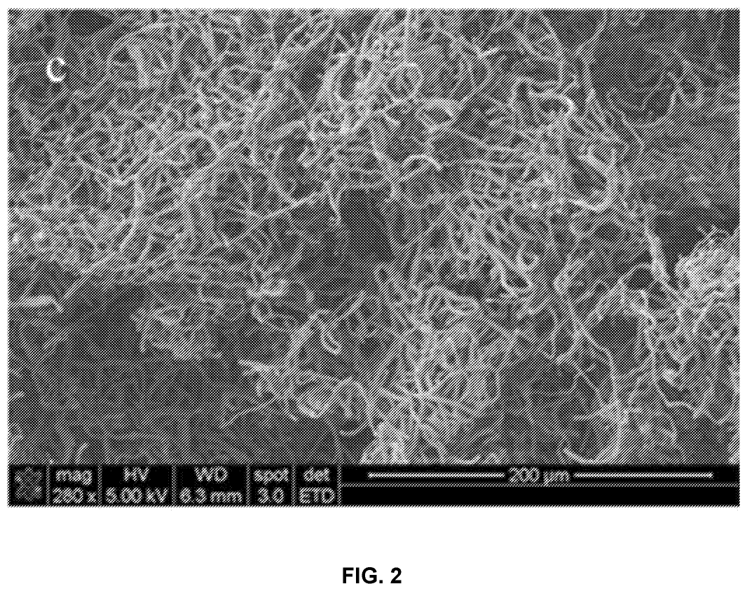

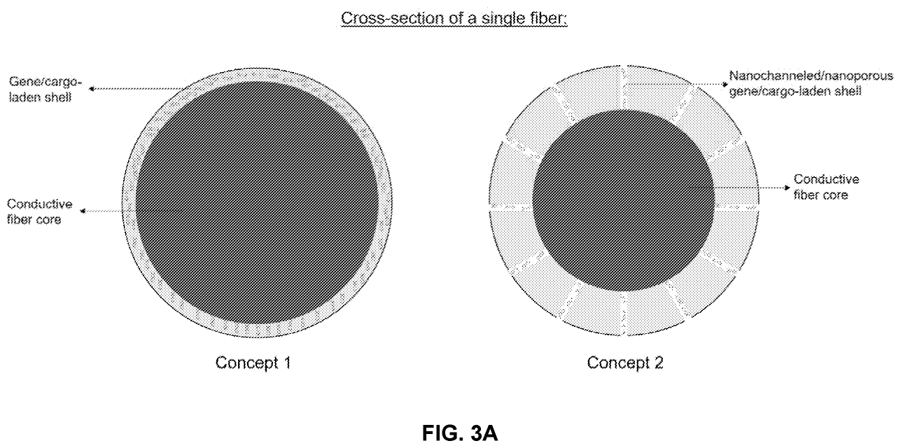

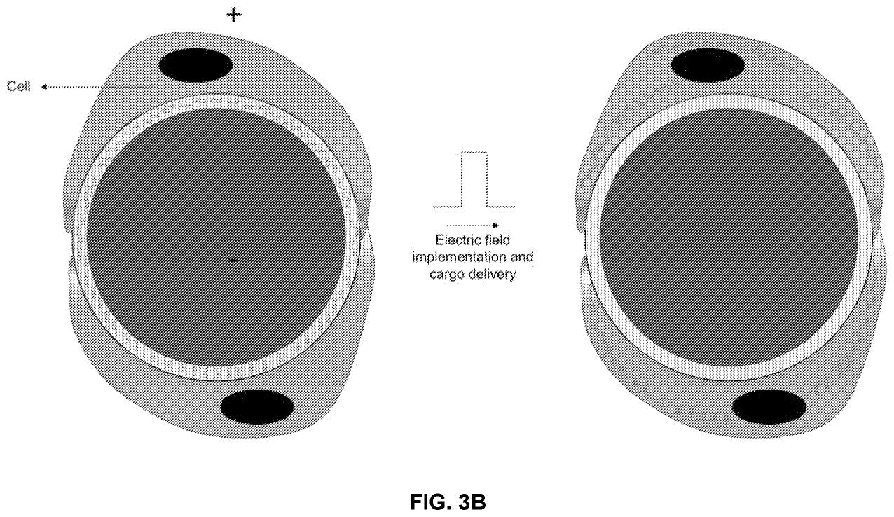

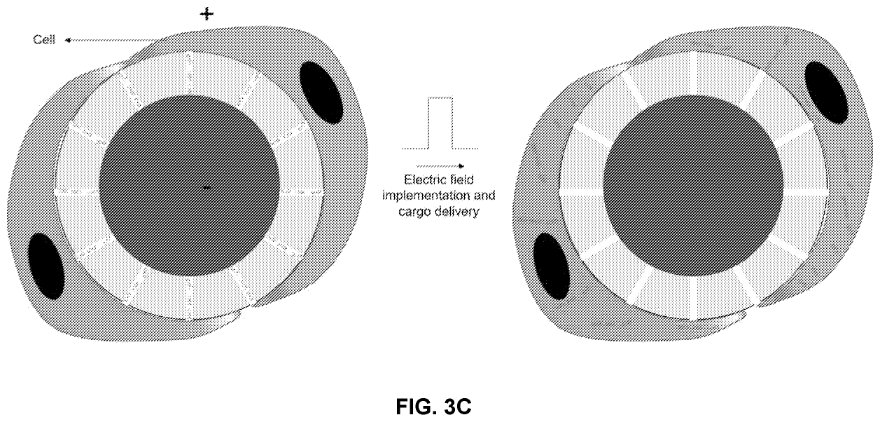

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

In some instances, units may be used herein that are non-metric or non-SI units. Such units may be, for instance, in U.S. Customary Measures, e.g., as set forth by the National Institute of Standards and Technology, Department of Commerce, United States of America in publications such as NIST HB 44, NIST HB 133, NIST SP 811, NIST SP 1038, NBS Miscellaneous Publication 214, and the like. The units in U.S. Customary Measures are understood to include equivalent dimensions in metric and other units (e.g., a dimension disclosed as “1 inch” is intended to mean an equivalent dimension of “2.5 cm”; a unit disclosed as “1 pcf” is intended to mean an equivalent dimension of 0.157 kN/m³; or a unit disclosed 100° F. is intended to mean an equivalent dimension of 37.8° C.; and the like) as understood by a person of ordinary skill in the art.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

The term “wound,” as used herein, refers to physical disruption of the continuity or integrity of tissue structure. “Wound healing” refers to the restoration of tissue integrity. It will be understood that this can refer to a partial or a full restoration of tissue integrity. Treatment of a wound thus refers to the promotion, improvement, progression, acceleration, or otherwise advancement of one or more stages or processes associated with the wound healing process.

“Parenteral administration”, as used herein, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

“Topical administration”, as used herein, means the non-invasive administration to the skin, orifices, or mucosa. Topical administrations can be administered locally, i.e. they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

In some aspects, the compounds, compositions, and methods disclosed herein can be utilized with or on a subject in need of treatment, which can also be referred to as “in need thereof.” As used herein, the phrase “in need thereof means that the subject has been identified as having a need for the particular method or treatment and that the treatment has been given to the subject for that particular purpose.

The term “subject” as used herein includes, but is not limited to, humans, nonhuman vertebrates, and animals such as wild, domestic, and farm animals. Preferably, the term “subject” refers to mammals. More preferably, the term “subject” refers to humans.

A “therapeutically effective amount” or “effective amount” of a composition is a predetermined amount calculated to achieve the desired effect (e.g., amount of bioactive agent to treat a wound). The core-shell fibers described herein are effective over a wide dosage range. It will be understood that the effective amount administered will be determined by the physician, veterinarian, or other medical professional in the light of the relevant circumstances including the condition to be treated, the choice of compound to be administered, and the chosen route of administration, and therefore the dosage ranges described herein are not intended to limit the scope of the disclosure in any way.

The terms “treat,” “treated,” or “treating” as used herein refer to both therapeutic treatment and prophylactic or preventative measures, wherein the object is to prevent or slow down (lessen) or entirely reverse (eradicate) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; remission (whether partial or total), whether detectable or undetectable, or enhancement or improvement of the condition, disorder, or disease; and eradication of the condition, disorder, or disease. Treatment includes eliciting a clinically significant response without excessive levels of side effects. Treatment also includes prolonging survival as compared to expected survival if not receiving treatment.

### Core-Shell Fibers and Methods of Making

A variety of core-shell fibers are provided herein as well as fiber clusters, textiles, meshes, mats, and various formulations thereof that can be used for the efficient delivery of bioactive agents. In some aspects, core-shell fibers are provided that include (i) a central core that is electrically conductive having an exterior surface, wherein the core comprises a first polymer and an electroconductive material; (ii) a shell adjacent to the exterior surface of the core, the shell comprising a second polymer.

A variety of different polymers can be used to produce the core and shell of the fibers described herein. In one aspect, the first polymer in the core can be the same polymers as the second polymer in the shell. In another aspect, the first polymer and the second polymer are different polymers. In other aspects, the first and second polymer are biocompatible.

In one aspect, the first and second polymer can be a synthetic or semi-synthetic polymers such as, without limitation, polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.

In another aspect, the first and second polymer can be a natural polymer such as, for example, fibronectin, collagen, gelatin, hyaluronic acid, chitosan, or combinations thereof. It may be understood that the first polymer and/or second polymer may also include a combination of synthetic polymers and naturally occurring polymers in any combination or compositional ratio.

In another aspect, the first and second polymer can be a biocompatible polymer selected from the group consisting of polyalkylene glycols such as poly(ethylene glycol) and poly(propylene glycol); aliphatic polyesters based on hydroxyalkanoic acids, such as poly(lactic acid), poly(glycolic acid), poly(e-caprolactone) and related copolymers; poly(methyl methacrylate); poly(ethylene vinyl acetate); poly(2-hydroxyethyl methacrylate); polyvinylpyrrolidone; copolymers thereof; and blends thereof.

In certain aspects, the first polymer and second polymer can be formulated as solutions prior to forming the core-shell fiber. For example, the first polymer and second polymer can independently be formulated with one or more solvents to produce compositions or solutions suitable for electrospinning fibers. In one aspect, the solvent can be acetone, dimethylformamide, dimethylsulfoxide, N-methylpyrrolidone, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof.

When formulated with solvents, the amount of the first polymer and second polymer can vary. In one aspect, the first polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %). In another aspect, the second polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %).

The shell of the core-fiber includes an electroconductive material in addition to the first polymer. An electroconductive material is any material capable of conducting electricity. In one aspect, the electroconductive material is an electroconductive polymer. Examples of electroconductive polymers include, but are not limited to, polyaniline, poly(pyrrole)s, oxidized polyacetylenes, poly(fluorene)s, polyphenylenes, polypyrenes, polyazulenes, polynaphthalenes, poly(p-phenylene vinylene), polycarbazoles, polyindoles, polyazepines, poly(thiophene)s, poly(3,4-ethylenedioxythiophene), poly(p-phenylene sulfide), poly(naphthalene vinylene)s, poly(3,4-ethylenedioxythiophene)-poly(styrenesulfonate), poly(3,4-ethylenedioxythiophene)-block-poly(ethylene glycol), or any combination thereof. In one aspect, the electroconductive polymer can include one polymer or two or more polymers.

In one aspect, the electroconductive material is an electroconductive metal. Examples of electroconductive metals include, but are not limited to, tantalum, gold, niobium, silver, copper, aluminum, iron, zinc, molybdenum, lithium, nickel, palladium, platinum, tungsten, tin, rhodium, Iridium, or any combination thereof. In one aspect, the electroconductive metal can include one metal or two or more metals. In one aspect, the electroconductive metal can be nanoparticles having an average diameter of about 1 nm, 10 nm, 50 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm, where any value can be a lower and upper endpoint of a range (e.g., 100 nm to 500 nm).

In one aspect, the electroconductive material can include a combination of one or more electroconductive polymers with one or more electroconductive metals. For example, the electroconductive material can be a combination of polyaniline and tantalum particles.

When formulated with solvents, the amount of the first polymer and electroconductive material can vary. In one aspect, the first polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %) and the electroconductive polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %). In another aspect, the first polymer is from 1 wt % to 50 wt % of the composition, or about 1 wt %, 5 wt %, 10 wt %, 15 wt %, 20 wt %, 25 wt %, 30 wt %, 35 wt %, 40 wt %, 45 wt %, or 50 wt %, where any value can be a lower and upper endpoint of a range (e.g., 5 wt % to 25 wt %) and the electroconductive metal is from 1 wt % to 90 wt % of the composition, or about 1 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or 90 wt %, where any value can be a lower and upper endpoint of a range (e.g., 5 wt % to 25 wt %).

In one aspect, the weight ratio of the first polymer to the electroconductive polymer in the core-shell fiber is from 2:1 to 1:2, or is 2:1, 1.5:1, 1;1, 1:1.5, or 1:2, where any value can be a lower and upper endpoint of a range (e.g., 1.5:1 to 1:1.5). In another aspect, the weight ratio of the first polymer to the electroconductive metal in the core-shell fiber is from 1:10 to 1:1, or is 1:10, 1:9, 1:8, 1:7, 1:6, 1:5, 1:4, 1:3, 1:2, or 1:1, where any value can be a lower and upper endpoint of a range (e.g., 1:10 to 1:7).

The core-shell fibers described herein are electrospun fibers. Electrospinning is a method of spinning a polymer fiber or polymer nanofiber from a polymer solution by applying a high DC voltage potential between the polymer solution (or polymer injection system containing the polymer solution) and a receiving surface for the electrospun polymer nanofibers. The voltage potential may include voltages less than or equal to about 15 kV. The polymer may be ejected by a polymer injection system at a flow rate of less than or equal to about 5 mL/h. As the polymer solution travels from the polymer injection system toward the receiving surface, it may be elongated into sub-micron diameter electrospun polymer nanofibers, typically in the range of about 0.1 μm to about 10 μm. Some non-limiting examples of electrospun polymer nanofiber diameters may include about 0.1 μm, about 0.2 μm, about 0.5 μm, about 1 μm, about 2 μm, about 5 μm, about 10 μm, about 20 μm, or ranges between any two of these values (including endpoints). A polymer injection system may include any system configured to eject some amount of a polymer solution into an atmosphere to permit a flow of the polymer solution from the injection system to the receiving surface. In some non-limiting examples, the injection system may deliver a continuous stream of a polymer solution to be formed into a polymer nanofiber. In alternative examples, the injection system may be configured to deliver intermittent streams of a polymer to be formed into multiple polymer nanofibers. In one embodiment, the injection system may include a syringe under manual or automated control. In another embodiment, the injection system may include multiple syringes under individual or combined manual or automated control. In some examples, a multi-syringe injection system may include multiple syringes, each syringe containing the same polymer solution. In alternative examples, a multi-syringe injection system may include multiple syringes, each syringe containing a different polymer solution.

The receiving surface may move with respect to the polymer injection system, or the polymer injection system may move with respect to the receiving surface. In some embodiments, the receiving surface may move with respect to the polymer injection system under manual control. In other embodiments, the surface may move with respect to the polymer system under automated control. In such embodiments, the receiving surface may be in contact with or mounted upon a support structure that may be moved using one or more motors or motion control systems. In some non-limiting examples, the surface may be a roughly cylindrical surface configured to rotate about a long axis of the surface. In some other non-limiting examples, the surface may be a flat surface that rotates about an axis approximately coaxial with the polymer fiber ejected by the polymer injection system. In yet some other non-limiting examples, the surface may be translated in one or more of a vertical direction and a horizontal direction with respect to the polymer nanofiber ejected by the polymer injection system. It may be further recognized that the receiving surface of the polymer nanofiber may move in any one direction or combination of directions with respect to the polymer nanofiber ejected by the polymer injection system. The pattern of the electrospun polymer nanofiber deposited on the receiving surface may depend upon the one or more motions of the receiving surface with respect to the polymer injection system. In one non-limiting example, a roughly cylindrical receiving surface, having a rotation rate about its long axis that is faster than a translation rate along a linear axis, may result in a roughly helical deposition of an electrospun polymer fiber forming windings about the receiving surface. In an alternative example, a receiving surface having a translation rate along a linear axis that is faster than a rotation rate about a rotational axis, may result in a roughly linear deposition of an electrospun polymer fiber along a liner extent of the receiving surface.

In some embodiments, the receiving surface may be coated with a non-stick material, such as, for example, aluminum foil, a stainless steel coating, polyteirafluoroethylene, or a combination thereof, before the application of the electrospun polymer nanofibers. The receiving surface, such as a mandrel, may be fabricated from aluminum, stainless steel, polytetrafluoroethylene, or a combination thereof to provide a nonstick surface on which the electrospun nanofibers may be deposited. In other embodiments, the receiving surface may be coated with a simulated cartilage or other supportive tissue. In some non-limiting examples, the receiving surface may be composed of a planar surface, a circular surface, an irregular surface, and a roughly cylindrical surface. One embodiment of a roughly cylindrical surface may be a mandrel. A mandrel may take the form of a simple cylinder, or may have more complex geometries. In some non-limiting examples, the mandrel may take the form of a hollow bodily tissue or organ. In some non-limiting examples, the mandrel may be matched to a subject's specific anatomy.

In one aspect, the core-shell fiber is produced by electrospinning a concentric composition comprising an inner first composition comprising the first polymer and an electroconductive polymer and a second outer composition comprising the second polymer. In one aspect, a higher gauge needle can be inserted into a lower gauge needle. A syringe with the first polymer and electroconductive material will be injected into the higher gauge needle concurrently while the second polymer is injected into the lower gauge needle during electrospinning to produce the core-shell fiber. Upon electrospinning, the shell layer is adjacent to (i.e., intimate contact with) the core such that a shell is formed around the entire core. The Examples provide non-limiting methods for making the core-shell fibers described herein.

The thickness of the shell layer can vary depending upon the application of the core-shell fibers. For example, where it is desirable to deliver larger amounts of bioactive agent, the thickness of the shell can be increased to hold more of the agent. In one aspect, the shell has a thickness of about 10 nm to about 20 μm, or 10 nm, 50 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, where any value can be a lower and upper endpoint of a range (e.g., 800 nm to 15 μm).

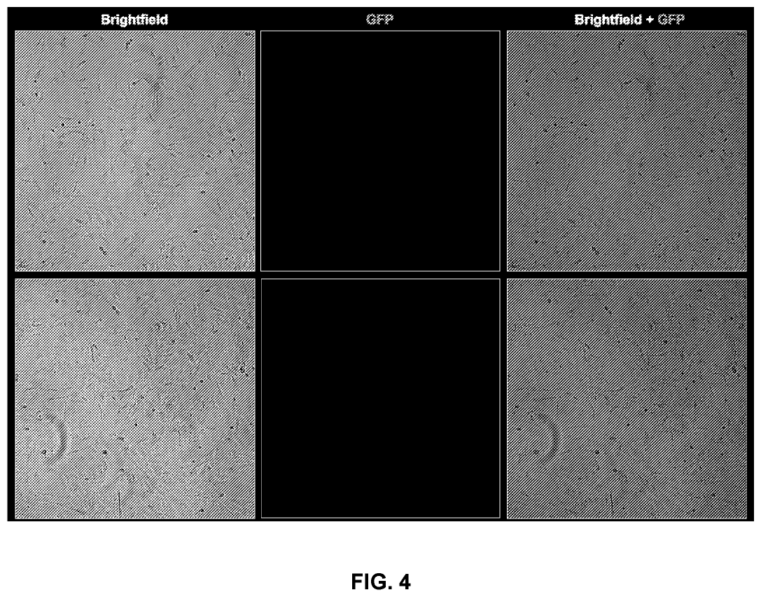

In certain aspects, the shell comprises a plurality of nanochannels. FIG. 4A depicts nanochannels in the fiber. In one aspect, the nanochannels have an average diameter of about 10 nm to about 1,000 nm, or 10 nm, 50 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, or 1,000 nm, where any value can be a lower and upper endpoint of a range (e.g., 200 nm to 750 nm). The nanochannels can be made by a variety of techniques. In one aspect, the second polymer can be formulated in a solvent that is immiscible with water. Upon electrospinning in a humid environment and subsequent evaporation of the solvent, nanochannels are produced.

In one aspect, the core-shell fibers described herein can be produced by electrospinning a plurality of fiber fragments. As used herein, the term “fragment” refers to a portion of a particular fiber. In some aspects, a fragment can have an average length of about 1 μm to about 1,000 μm, and an average diameter of about 0.1 μm to about 10 μm. Some non-limiting examples of average fragment lengths may include an average length of about 1 μm, an average length of about 5 μm, an average length of about 10 μm, an average length of about 20 μm, an average length of about 30 μm, an average length of about 40 μm, an average length of about 50 μm, an average length of about 75 μm, an average length of about 90 μm, an average length of about 95 μm, an average length of about 100 μm, an average length of about 105 μm, an average length of about 125 μm, an average length of about 150 μm, an average length of about 200 μm, an average length of about 300 μm, an average length of about 400 μm, an average length of about 500 μm, an average length of about 600 μm, an average length of about 700 μm, an average length of about 800 μm, an average length of about 900 μm, an average length of about 1000 μm, or ranges between any two of these values (including endpoints).

Some non-limiting examples of average fragment diameters may include an average diameter of about 0.1 μm, an average diameter of about 0.5 μm, an average diameter of about 1 μm, an average diameter of about 2 μm, an average diameter of about 3 μm, an average diameter of about 4 μm, an average diameter of about 5 μm, an average diameter of about 6 μm, an average diameter of about 7 μm, an average diameter of about 8 μm, an average diameter of about 9 μm, an average diameter of about 10 μm, or ranges between any two of these values (including endpoints). When combined with a carrier medium, the resulting mixture may include from about 1 fragment per mm³ to about 100,000 fragments per mm³. Some non-limiting examples of mixture densities may include about 2 fragments per mm³, about 100 fragments per mm³, about 1,000 fragments per mm³, about 2,000 fragments per mm³, about 5,000 fragments per mm³, about 10,000 fragments per mm³, about 20,000 fragments per mm³, about 30,000 fragments per mm³, about 40,000 fragments per mm³, about 50,000 fragments per mm³, about 60,000 fragments per mm³, about 70,000 fragments per mm³, about 80,000 fragments per mm³, about 90,000 fragments per mm³, about 100,000 fragments per mm³, or ranges between any two of these values (including endpoints).

As used herein, the term “cluster” refers to an aggregate of fiber fragments, or a linear or curved three-dimensional group of fiber fragments. Clusters may have a range of shapes. Non-limiting examples of cluster shapes may include spherical, globular, ellipsoidal, and flattened cylinder shapes. Clusters may have, independently, an average length of about 1 μm to about 1000 μm, an average width of about 1 μm to about 1000 μm, and an average height of about 1 μm to about 1000 μm. It may be appreciated that any cluster dimension, such as length, width, or height, is independent of any other cluster dimension. Some non-limiting examples of average cluster dimensions include an average dimension (length, width, height, or other measurement) of about 1 μm, an average dimension of about 5 μm, an average dimension of about 10 μm, an average dimension of about 20 μm, an average dimension of about 30 μm, an average dimension of about 40 μm, an average dimension of about 50 μm, an average dimension of about 75 μm, an average dimension of about 90 μm, an average dimension of about 95 μm, an average dimension of about 100 μm, an average dimension of about 105 μm, an average dimension of about 110 μm, an average dimension of about 150 μm, an average dimension of about 200 μm, an average dimension of about 300 μm, an average dimension of about 400 μm, an average dimension of about 500 μm, an average dimension of about 600 μm, an average dimension of about 700 μm, an average dimension of about 800 μm, an average dimension of about 900 μm, an average dimension of about 1000 μm, or ranges between any two of these values (including endpoints), or independent combinations of any of these ranges of dimensions. Clusters may include an average number of about 2 to about 1000 fiber fragments. Some non-limiting examples of average numbers of fiber fragments per cluster include an average of about 2 fiber fragments per cluster, an average of about 5 fiber fragments per cluster, an average of about 10 fiber fragments per cluster, an average of about 20 fiber fragments per cluster, an average of about 30 fiber fragments per cluster, an average of about 40 fiber fragments per cluster, an average of about 50 fiber fragments per cluster, an average of about 60 fiber fragments per cluster, an average of about 70 fiber fragments per cluster, an average of about 80 fiber fragments per cluster, an average of about 90 fiber fragments per cluster, an average of about 100 fiber fragments per cluster, an average of about 110 fiber fragments per cluster, an average of about 200 fiber fragments per cluster, an average of about 300 fiber fragments per cluster, an average of about 400 fiber fragments per cluster, an average of about 500 fiber fragments per cluster, an average of about 600 fiber fragments per cluster, an average of about 700 fiber fragments per cluster, an average of about 800 fiber fragments per cluster, an average of about 900 fiber fragments per cluster, an average of about 1000 fiber fragments per cluster, or ranges between any two of these values (including endpoints). In some embodiments, a composition may contain a plurality of clusters.

In some aspect, the core-shell fibers described herein can be used to produce a textile. The term “textile” is defined herein as a spun, woven, or otherwise fabricated material comprising the core-shell fibers described herein. The textile can include meshes, mats, and the like. In the case when the core-shell fiber is electrospun to produce a mesh or mat, the fibers can be aligned or substantially aligned (i.e., greater than 95% aligned). In other aspects, the fibers can be randomly aligned.

In some aspects, the core-shell fibers may be wound about a mandrel, as threads are wound around a bobbin. In some aspects, the core-shell fibers may be deposited, in an essentially parallel manner, along a linear dimension of a mandrel or other surface form. In some embodiments, winding the textile may use electrospinning techniques. The term “pore size” is thus defined herein as being the diameter of introduced pores, pockets, voids, holes, spaces, etc. introduced in an unmeshed structure such as a block polymer, polymer sheet, or formed polymer scaffold, and is specifically distinguished from “mesh size” as disclosed herein. As used herein, the term “mesh size” is the number of openings in a textile per linear measure. For example, if the textile has 1200 openings per linear millimeter, the textile is defined 1200 mesh (e.g., sufficient to allow a 12 micron red blood cell to pass), which is easily convertible between imperial and metric units. A mesh size may be determined based on the number of fibers having a specified average diameter and an average opening size between adjacent fibers along a specified linear dimension. Thus, a textile composed of 10 um average diameter fibers having 10 μm average diameter openings between adjacent fibers may have about 50 total openings along a 1 mm length and may therefore be defined as a 50 mesh textile.

In some aspects, the core-shell fiber is spun over a mandrel so as to form a textile roll or tube. In some embodiments, the thickness of the textile roll or tube may be regulated by changing the number of rotations of the mandrel over time while the textile roll or tube collects the fiber. In certain other embodiments, the biocompatible textile has a mesh size of about 1 opening per mm to about 20 openings per mm. Some non-limiting examples of mesh sizes may include about 1 opening per mm, about 2 openings per mm, about 4 openings per mm, about 6 openings per mm, about 8 openings per mm, about 10 openings per mm, about 15 openings per mm, about 20 openings per mm, or ranges between any two of these values (including endpoints). In some embodiments, the mesh size of the spun textile may be about 20 openings per mm to about 500 openings per mm. Some non-limiting examples of mesh sizes may include about 20 openings per mm, about 40 openings per mm, about 60 openings per mm, about 80 openings per mm, about 100 openings per mm, about 200 openings per mm, about 300 openings per mm, about 400 openings per mm, about 500 openings per mm, or ranges between any two of these values (including endpoints). In other embodiments, the mesh size may be about 500 openings per mm to about 1000 openings per mm. Some non- limiting examples of mesh sizes may include about 500 openings per mm, about 600 openings per mm, about 700 openings per mm, about 800 openings per mm, about 1000 openings per mm, or ranges between any two of these values (including endpoints). In some embodiments, the mesh size of the textile may be regulated by changing the speed and direction by which the fiber is deposited onto the mandrel, such as, by example, moving the position and direction in which the thread is spun onto the textile roll or tube.

### Bioactive Agents

The core-shell fibers can be loaded with any bioactive agent (e.g., therapeutic, prophylactic, or diagnostic agents) compatible with the shell, i.e. with any agent that can be loaded into the polymers of the shell. Preferred agents that can be loaded include genes useful for therapeutic, prophylactic, or diagnostic effect.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

As mentioned previously, the agents can include genes and other nucleic acids. The agents can include an aptamer such as RNA, DNA or an artificial nucleic acid. The agent can be an anionic proteins, protein analogues, or nucleic acids.

The terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general and unless otherwise specified, an analogue of a particular nucleotide has the same base-pairing specificity; i.e., an analogue of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. Antisense is a polynucleotide that interferes with the function of DNA and/or RNA. The term nucleic acids refers to a string of at least two base-sugar-phosphate combinations. Natural nucleic acids have a phosphate backbone, artificial nucleic acids may contain other types of backbones, but contain the same bases. The term also includes PNAs (peptide nucleic acids), phosphorothioates, and other variants of the phosphate backbone of native nucleic acids.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

In one aspect, the bioactive agent can include one or more of a nucleic acid, a peptide, a polypeptide, a small molecule, a vaccine, vesicles isolated from cells that have been reprogrammed, and a combination thereof. The one or more therapeutic, prophylactic, or diagnostic agents can include one or more of genes such as LL37, laminin/collagen VII, and VEGF/EGF. The one or more bioactive agents can include RNA, DNA or an artificial nucleic acid. The one or more bioactive agents can include anionic proteins, protein analogues, or nucleic acids. The one or more therapeutic, prophylactic, or diagnostic agents can encode proteins selected from the group consisting of ETV2, FOXC2, and FLI1.

The bioactive agents can be loaded into/onto the core-shell fibers in several different methods. In one aspect, the bioactive agent can be added into solution of the second polymer before electrospinning. In another aspect, the bioactive agent can be adsorbed onto the fiber surface after electrospinning by soaking the fibers in a solution of the cargo. In another aspect, the bioactive agent can be embedded into the fibers via subcritical/supercritical carbon dioxide.

In one aspect, the bioactive agent is present only in the shell and not the core of the core-shell fibers. In certain aspects, when the shell comprises nanochannels, the bioactive agent is present in the nanochannels.

In certain aspects, when the core-shell fibers are electrospun into a mesh or mat, the bioactive agent can be adsorbed at different concentrations at different locations on the mat or mesh to produce a concentration gradient. Depending upon the application of the mesh or mat, different amounts of bioactive agent can be added to the mesh or mat. In other aspects, different bioactive agents can be applied to the mesh or mat at different locations. The nature and amount of the bioactive agent used and applied to the mesh or mat can vary depending upon the application.

### Cell Transfection

In some embodiments, the disclosed methods are used for ex vivo delivery of bioactive cargo to cells. This could be accomplished exposing the cells to the fibers, with or without the implementation of an electric field. To expose the cells to the fibers, the cells could be pre-adhered to a culture vessel, and the fibers could be introduced into the vessel. Alternatively, the cells could be allowed to adhere directly to the fibers. For suspension cells, the cells and fibers can be mixed at various ratios. Bioactive cargo transfer can happen passively, facilitated by the presence of the fibers and subsequent response of the cells to micro- and/or nano-structured materials. Bioactive cargo transfer can also be achieved and enhanced by applying an external electric field, which will focus the field lines directly on the conductive core, and enable nanoscale electroporation-based transfer of the cargo to the cells.

These methods can be used with any cell type suitable for transfection. Mammalian cells include Keratinizing Epithelial Cells, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet Stratified Barrier Epithelial Cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining bladder and urinary ducts), Exocrine Secretory Epithelial Cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), Clara cell of lung, Hormone Secreting Cells, Anterior pituitary cell secreting growth hormone, Anterior pituitary cell secreting follicle-stimulating hormone, Anterior pituitary cell secreting luteinizing hormone, Anterior pituitary cell secreting prolactin, Anterior pituitary cell secreting adrenocorticotropic hormone, Anterior pituitary cell secreting thyroid-stimulating hormone, Intermediate pituitary cell secreting melanocyte-stimulating hormone, Posterior pituitary cell secreting oxytocin, Posterior pituitary cell secreting vasopressin, Gut and respiratory tract cell secreting serotonin, Gut and respiratory tract cell secreting endorphin, Gut and respiratory tract cell secreting somatostatin, Gut and respiratory tract cell secreting gastrin, Gut and respiratory tract cell secreting secretin, Gut and respiratory tract cell secreting cholecystokinin, Gut and respiratory tract cell secreting insulin, Gut and respiratory tract cell secreting glucagon, Gut and respiratory tract cell secreting bombesin, Thyroid gland cell secreting thyroid hormone, Thyroid gland cell secreting calcitonin, Parathyroid gland cell secreting parathyroid hormone, Parathyroid gland oxyphil cell, Adrenal gland cell secreting epinephrine, Adrenal gland cell secreting norepinephrine, Adrenal gland cell secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, Mesangial cell of kidney, Epithelial Absorptive Cells (Gut, Exocrine Glands and Urogenital Tract), Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, Epididymal basal cell, Metabolism and Storage Cells, Hepatocyte (liver cell), White fat cell, Brown fat cell, Liver lipocyte, Barrier Function Cells (Lung, Gut, Exocrine Glands and Urogenital Tract), Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.), Epithelial Cells Lining Closed Internal Body Cavities, Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, Corneal endothelial cell, Ciliated Cells with Propulsive Function, Respiratory tract ciliated cell, Oviduct ciliated cell (in female), Uterine endometrial ciliated cell (in female), Rete testis cilated cell (in male), Ductulus efferens ciliated cell (in male), Ciliated ependymal cell of central nervous system (lining brain cavities), Extracellular Matrix Secretion Cells, Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other (nonepithelial) fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Contractile Cells, Red skeletal muscle cell (slow), White skeletal muscle cell (fast), Intermediate skeletal muscle cell, Muscle spindle—nuclear bag cell, Muscle spindle—nuclear chain cell, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, Blood and Immune System Cells, Erythrocyte (red blood cell), Megakaryocyte, Monocyte, Connective tissue macrophage (various types), Epidermal Langerhans cell, Osteoclast (in bone), Dendritic cell (in lymphoid tissues), Microglial cell (in central nervous system), Neutrophil, Eosinophil, Basophil, Mast cell, Helper T lymphocyte cell, Suppressor T lymphocyte cell, Killer T lymphocyte cell, IgM B lymphocyte cell, IgG B lymphocyte cell, IgA B lymphocyte cell, IgE B lymphocyte cell, Killer cell, Stem cells and committed progenitors for the blood and immune system (various types), Sensory Transducer Cells, Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), Proprioceptive primary sensory neurons (various types), Autonomic Neuron Cells, Cholinergic neural cell (various types), Adrenergic neural cell (various types), Peptidergic neural cell (various types), Sense Organ and Peripheral Neuron Supporting Cells, Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), Enteric glial cell, Central Nervous System Neurons and Glial Cells, Neuron cell (large variety of types, still poorly classified), Astrocyte glial cell (various types), Oligodendrocyte glial cell, Lens Cells, Anterior lens epithelial cell, Crystallin-containing lens fiber cell, Pigment Cells, Melanocyte, Retinal pigmented epithelial cell, Germ Cells, Oogonium/oocyte, Spermatocyte, Spermatogonium cell (stem cell for spermatocyte), Nurse Cells, Ovarian follicle cell, Sertoli cell (in testis), and Thymus epithelial cell. In some cases, the cell is microorganism, such as a bacteria, protist, or yeast. In some cases, the cell is a plant cell.

In some embodiments, the disclosed methods are used to manufacture extracellular vesicles (EVs). The disclosed EVs can in some embodiments be any that can be produced by a cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

The disclosed extracellular vesicles may be prepared by methods known in the art. For example, the disclosed extracellular vesicles may be prepared by expressing in a eukaryotic cell an mRNA that encodes the cell-targeting ligand. In some embodiments, the cell also expresses an mRNA that encodes a therapeutic cargo. The mRNA for the cell-targeting ligand and the therapeutic cargo may be expressed from vectors that are transfected into suitable production cells for producing the disclosed EVs. The mRNA for the cell-targeting ligand and the therapeutic cargo may be expressed from the same vector (e.g., where the vector expresses the mRNA for the cell-targeting ligand and the anti-inflammatory cargo from separate promoters), or the mRNA for the cell-targeting ligand and the therapeutic cargo may be expressed from separate vectors. The vector or vectors for expressing the mRNA for the cell-targeting ligand and the therapeutic cargo may be packaged in a kit designed for preparing the disclosed extracellular vesicles.

Also disclosed is a composition comprising an EV containing the disclosed targeting ligands. In some embodiments, the EV is loaded with a therapeutic cargo. Also disclosed is an EV producing cell engineered to secrete the disclosed EVs.

EVs, such as exosomes, are produced by many different types of cells including immune cells such as B lymphocytes, T lymphocytes, dendritic cells (DCs) and most cells. EVs are also produced, for example, by glioma cells, platelets, reticulocytes, neurons, intestinal epithelial cells and tumor cells. EVs for use in the disclosed compositions and methods can be derived from any suitable cell, including the cells identified above. Non-limiting examples of suitable EV producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells. EVs can also be obtained from autologous patient-derived, heterologous haplotype-matched or heterologous stem cells so to reduce or avoid the generation of an immune response in a patient to whom the exosomes are delivered. Any EV-producing cell can be used for this purpose.

EVs produced from cells can be collected from the culture medium by any suitable method. Typically a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters, gradient ultracentrifugation (for example, with sucrose gradient) or a combination of these methods.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

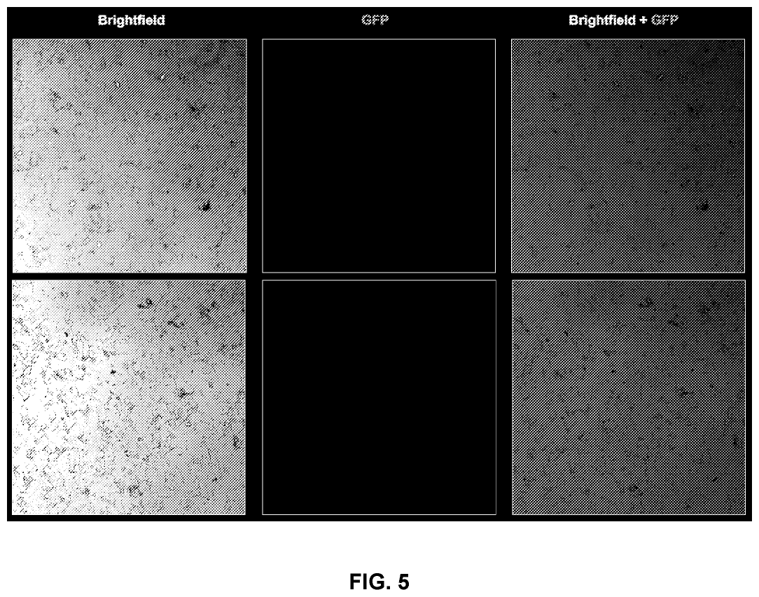

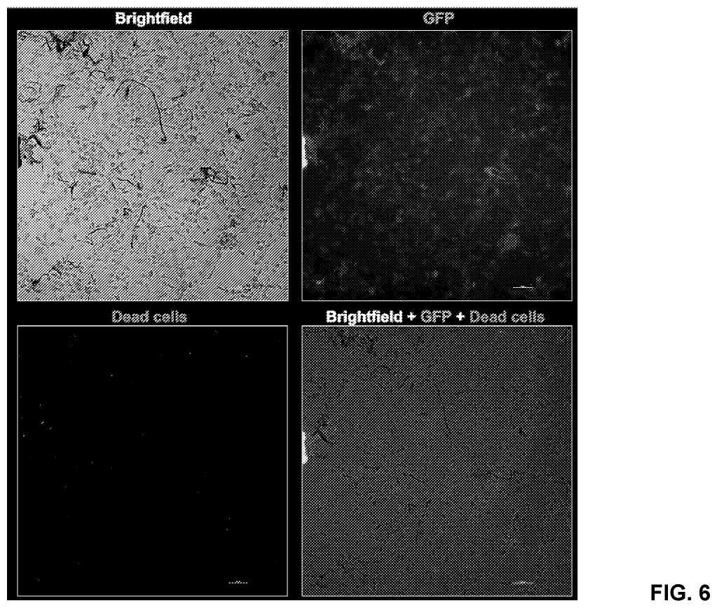

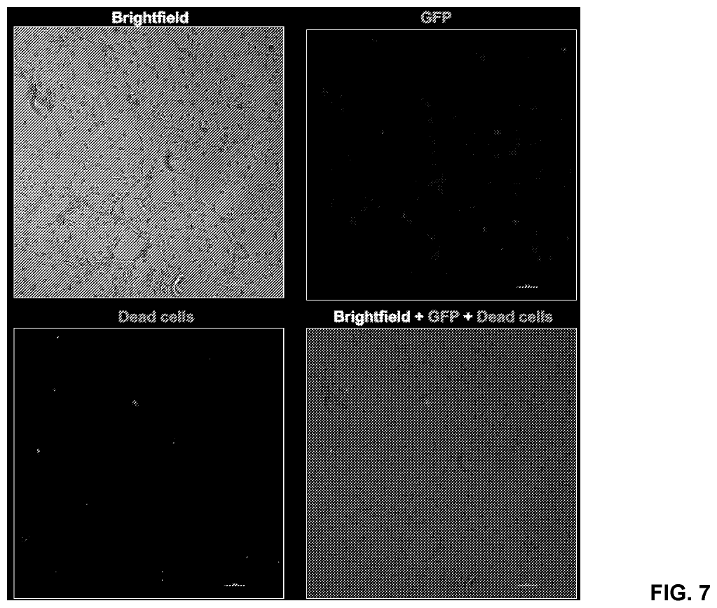

Cells incubated alone with GFP plasmid does not lead to successful transfection (as evidenced by the lack of GFP signal) (FIG. 4). Cells incubated with, fibers that had no, coating/shell with, GFP plasmid also do, not show any GFP, signal (FIG. 5). Cells incubated with fibers that had a coating/shell of GFP plasmid show successful transfer and expression of GFP. Ethidium homodimer was used to stain dead cells (red), to show that the procedure was not harmful to cells (FIG. 6). In cells incubated with GFP plasmid alone, no GFP signal is evident. More importantly, the number of dead cells seen in this control comparable to the number of dead cells we saw when they were incubated with fibers (FIG. 7).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An ex vivo method for delivering bioactive cargo to cells, comprising exposing the cells and bioactive cargo to electrospun core-shell fibers that comprise:
(i) a central core that is electrically conductive having an exterior surface, wherein the core comprises a first polymer and an electroconductive material;
(ii) a shell adjacent to the exterior surface of the core, the shell comprising a second polymer; and
(iii) one or more bioactive agents in the shell.

2. The method of claim 1, wherein the exposure occurs in the presence of an electric field.

3. The method of claim 1, wherein the cells are pre-adhered to a culture vessel, and wherein the electrospun core-shell fibers are introduced into the vessel.

4. The method of claim 1, wherein the cells are adhered to the electrospun core-shell fibers.

5. The method of claim 1, wherein the electroconductive material comprises an electroconductive polymer, an electroconductive metal, or a combination thereof.

6. The method of claim 1, wherein the electroconductive polymer comprises polyaniline, polyaniline, a poly(pyrrole), an oxidized polyacetylene, a poly(fluorene), a polyphenylenes, a polypyrene, a polyazulene, a polynaphthalene, a poly(p-phenylene vinylene), a polycarbazole, a polyindoles, a polyazepine, a poly(thiophene), a poly(3,4-ethylenedioxythiophene), a poly(p-phenylene sulfide), a poly(naphthalene vinylene), a poly(3,4-ethylenedioxythiophene)-poly(styrenesulfonate), a poly(3,4-ethylenedioxythiophene)-block-poly(ethylene glycol), or any combination thereof.

7.

8. The method of claim 1, wherein the weight ratio of the first polymer to the electroconductive polymer is from 2:1 to 1:2.

9. The method of claim 1, wherein the electroconductive metal comprises tantalum, gold, niobium, silver, copper, aluminum, iron, zinc, molybdenum, lithium, nickel, palladium, platinum, tungsten, tin, rhodium, Iridium, or any combination thereof.

10. The method of claim 1, wherein the electroconductive metal comprises a plurality of metal nanoparticles.

11. The method of claim 1, wherein the weight ratio of the first polymer to the electroconductive metal is from 1:10 to 1:1.

12. The method of claim 1, wherein the electroconductive material comprises a combination of one or more electroconductive polymers and one or more electroconductive metals.

13. The method of claim 1, wherein the first polymer is biocompatible.

14. The method of claim 1 wherein the first polymer comprises a synthetic polymer comprising polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.

15. The method of claim 1, wherein the first polymer comprises a natural polymer comprising fibronectin, collagen, gelatin, hyaluronic acid, chitosan, or any combination thereof.

16. The method of claim 1, wherein the first polymer comprises poly(e-caprolactone), polylactic acid (PLA), polyglycolic acid (PGA), polylactide-co-glycolide copolymer (PLGA), or any combination thereof.

17. The method of claim 1, wherein the core has an average diameter of about 100 nm to about 20 μm.

18. The method of claim 1, wherein the second polymer is biocompatible.

19. The method of claim 1, wherein the second polymer comprises a synthetic polymer comprising polyethylene terephthalate, a polyester, a polymethylmethacrylate, polyacrylonitrile, a silicone, a polyurethane, a polycarbonate, a polyether ketone ketone, a polyether ether ketone, a polyether imide, a polyamide, a polystyrene, a polyether sulfone, a polysulfone, a polycaprolactone (PCL), a polylactic acid (PLA), a polyglycolic acid (PGA), a polylactide-co-glycolide copolymer (PLGA), a polyglycerol sebacic, a polydiol citrate, a polyhydroxy butyrate, a polyether amide, a polydiaxanone, or any combination thereof.

20. The method of claim 1, wherein the second polymer comprises a natural polymer comprising fibronectin, collagen, gelatin, hyaluronic acid, chitosan, or any combination thereof.

21. The method of claim 1, wherein the second polymer comprises poly(e-caprolactone), polylactic acid (PLA), polyglycolic acid (PGA), polylactide-co-glycolide copolymer (PLGA), or any combination thereof.

22. The method of claim 1, wherein the first polymer and the second polymer are the same polymer.

23. The method of claim 1, wherein the first polymer and the second polymer are the different polymers.

24. The method of claim 1, wherein the shell has a thickness of about 10 nm to about 20 μm.

25. The method of claim 1, wherein the shell comprises a plurality of nanochannels.

26. The method of claim 1, wherein the nanochannels have an average diameter of about 10 nm to about 1,000 nm.

27. The method of claim 1 polypeptide, a small molecule, a vaccine, vesicles isolated from cells that have been reprogrammed, and any combination thereof.

28. The method of claim 1, wherein the one or more bioactive agents comprise one or more of genes such as LL37, laminin/collagen VII, and VEGF/EGF.

29. The method of claim 1, wherein the fibers are continuous.
